(19) United States
(12) Patent Application Publication
(10) Pub. No.: US 2012/0279505 A1
(43) Pub. Date: Nov. 8, 2012

(54) TRACHEAL TUBE COMPRISING TRACHEA SEALING PORTION

(75) Inventor: Jin Hee Kim, Seoul (KR)

(73) Assignee: SEOUL NATIONAL UNIVERSITY BUNDANG HOSPITAL, Seongnam-si (KR)

(21) Appl. No.: 13/478,050

(22) Filed: May 22, 2012

Related U.S. Application Data
(63) Continuation of application No. PCT/KR2011/001096, filed on Feb. 18, 2011.

(30) Foreign Application Priority Data
Feb. 19, 2010 (KR) 10-2010-0015402

(51) Int. Cl. A61M 16/04

(52) U.S. Cl. 128/207.15

(57) ABSTRACT

The present invention provides a tracheal tube comprising: a hollow tube inserted into the trachea; a first volume expansion portion for encompassing one side of the hollow tube; a second volume expansion portion positioned below the first volume expansion portion; a first conduit for expanding the first volume expansion portion and the second volume expansion portion; and a second conduit for supplying media for sealing to a trachea sealing portion positioned between the first volume expansion portion and the second volume expansion portion. The tracheal tube of the present invention can reduce the pressure of two cuffs which are the volume expansion portions, and can effectively improve sealing by supplying media to the trachea sealing portion positioned between the two cuffs. The tracheal tube of the present invention can reduce pulmonary aspiration and can thus considerably prevent ventilator-associated pneumonia (VAP).

### CROSS-REFERENCE TO RELATED APPLICATION

This is a continuation of International Application No. PCT/KR2011/001096 filed on Feb. 18, 2011, which claims the priority to Korean Application No. 10-2010-0015402 filed Feb. 19, 2010, which applications are incorporated herein be reference.

### TECHNICAL FIELD

The present invention relates to a tracheal tube including a trachea sealing portion and a method for manufacturing the same.

### BACKGROUND ART

Treatment of a patient in need of mechanical ventilation frequently requires that an endotracheal tube be inserted into the trachea through the patient's mouth or nose and connected to a ventilator, which mechanically delivers air into the lungs through the tube periodically. An inflatable cuff is provided on an end of the tube and is configured to contact the inner wall of the trachea when inflated.

Such a use of an endotracheal tube, however, causes a number of problems in connection with secretions that gradually pass between the inner surface of the trachea and the outer surface of the cuff. Conventional tracheal tubes of the HVLP (High Volume Low Pressure) type are 1.5-2 times as large as the diameter of human tracheae and inevitably cause endotracheal longitudinal folds, which may result in pulmonary aspiration. This is one of the commonest reasons of VAP (Ventilator-Associated Pneumonia) occurring in ICU rooms.

Prevention of the VAP requires that secretions accumulated in the area above the cuff and below the glottis be discharged. Conventional approaches to tackle these problems include adjusting the pressure of the cuff, using a thin material for the cuff, and suctioning secretions between the trachea inner wall and the endotracheal tube to the outside.

The present invention has been made, in view of the above-mentioned problems, to prevent pulmonary aspiration, which is caused by endotracheal folds, while applying less pressure to the trachea.

### SUMMARY OF THE DISCLOSURE

An aspect of the present invention is to provide a tracheal tube including a trachea sealing portion.

Another aspect of the present invention is to provide a method for manufacturing the tracheal tube.

Another aspect of the present invention is to provide a method for preventing endotracheal aspiration using the tracheal tube.

In accordance with an aspect of the present invention, there is provided a tracheal tube including:

a hollow tube 100 inserted into trachea;

a first volume inflator 10 surrounding one side of the hollow tube 100;

a second volume 20 inflator positioned under the first volume inflator 10;

a first conduit 40 adapted to inflate the first and second volume inflators 10 and 20; and

a second conduit 50 adapted to supply a sealing medium 80 to a trachea sealing portion positioned between the first and second volume inflators 10 and 20.

The second conduit 50 may be positioned outside the hollow tube 100, in a section of the hollow tube 100, or inside the hollow tube 100.

When the second conduit 50 is positioned in a section of the hollow tube 100 or inside the hollow tube 100, a sealing medium passage hole 30 may be formed through the trachea sealing portion.

In another specific embodiment of the present invention, there is provided a tracheal tube including:

a hollow tube 100 inserted into trachea;

a first volume inflator 10 surrounding one side of the hollow tube 100;

a second volume inflator 20 positioned under the first volume inflator 10;

a first conduit adapted to inflate the first volume inflator 10;

a second conduit adapted to inflate the second volume inflator 20; and

a third conduit adapted to supply a sealing medium 80 to a trachea sealing portion positioned between the first and second volume inflators 10 and 20.

The third conduit may be positioned outside the hollow tube 100, in a section of the hollow tube 100, or inside the hollow tube 100. In addition, when the third conduit is positioned in a section of the hollow tube 100 or inside the hollow tube 100, a sealing medium passage hole may be formed through the trachea sealing portion.

The tracheal tube may further include a volume controller positioned on the other side of the tube and adapted to inflate the volume of the first or second volume inflator 10 or 20.

A volume inflation medium passage hole 31 may be formed through one side of the tube so that a volume inflation medium supplied from the volume controller can flow into the first or second volume inflator 10 or 20.

Preferably, the first or second volume inflator 10 or 20 is a cuff.

In a specific embodiment, the first or second volume inflator 10 or 20 may have a cylindrical shape.

During volume inflation, the first or second volume inflator 10 or 20 preferably has a diameter of 0.5-3.0 cm, but the present invention is not limited thereto.

During volume inflation, the first or second volume inflator 10 or 20 preferably has a length of 0.5-3.0 cm, but the present invention is not limited thereto.

The interval between the first and second volume inflators 10 and 20 is preferably 0.2-1.0 cm, but the present invention is not limited thereto.

The volume controller may include a volume regulator 60 adapted to regulate the flow direction of the volume inflation medium introduced through the first conduit; and a volume provider 70 adapted to introduce the volume inflation medium through the first conduit.

The volume provider 70 may be a syringe, but is not limited thereto.

The volume inflation medium may be gas or liquid.

The sealing medium 80 may be gel, jelly, or gas.

In accordance with another aspect of the present invention, there is provided a method for manufacturing a tracheal tube, the method including the steps of:

(i) preparing a hollow tube 100;

(ii) positioning a first volume inflator 10 on one side of the hollow tube 100;

(iii) positioning a second volume inflator 20 under the first volume inflator 10;

(iv) positioning a first conduit 40 inside the hollow tube 100, the first conduit 40 being adapted to inflate the first and second volume inflators 10 and 20; and

(v) positioning a second conduit 50 adapted to supply a sealing medium 80 to a trachea sealing portion between the first and second volume inflators 10 and 20.

In step (v), the second conduit 50 may be positioned outside the hollow tube 100, in a section of the hollow tube 100, or inside the hollow tube 100.

When the second conduit 50 is positioned in a section of the hollow tube 100 or inside the hollow tube 100, a sealing medium passage hole 30 may be formed through the trachea sealing portion.

In another specific embodiment of the present invention, there is provided a method for manufacturing a tracheal tube, the method including the steps of:

(i) preparing a hollow tube 100;

(ii) positioning a first volume inflator 10 on one side of the hollow tube 100;

(iii) positioning a second volume inflator 20 under the first volume inflator 10;

(iv) positioning a first conduit inside the hollow tube 100, the first conduit being adapted to inflate the first volume inflator 10;

(v) positioning a second conduit inside the hollow tube 100, the second conduit being adapted to inflate the second volume inflator 20; and

(vi) positioning a third conduit adapted to supply a sealing medium 80 to a trachea sealing portion between the first and second volume inflators 10 and 20.

In step (vi), the third conduit may be positioned outside the hollow tube 100, in a section of the hollow tube 100, or inside the hollow tube 100.

When the third conduit is positioned in a section of the hollow tube 100 or inside the hollow tube 100, a sealing medium passage hole 30 may be formed through the trachea sealing portion.

The method may further include a step of positioning a volume controller on the other side of the tube, the volume controller being adapted to inflate the volume of the first or second volume inflator 10 or 20.

A volume inflation medium passage hole 31 may be formed through one side of the tube so that a volume inflation medium supplied from the volume controller can flow into the first or second volume inflator 10 or 20.

In accordance with another aspect of the present invention, there is provided a method for preventing endotracheal aspiration by providing a medium through the conduits of the tracheal tube.

The tracheal tube according to the present invention can reduce the pressure of volume inflators, i.e. two cuffs, and supply a medium to a trachea sealing portion, which is positioned between the two cuffs, to improve the sealing effect. The tracheal tube according to the present invention can reduce pulmonary aspiration, and thus prevent VAP to a considerable extent. The tracheal tube according to the present invention is applicable to all types of patients in need of tracheal tubes, such as a patient in a serious condition requiring mechanical ventilation, a patient requiring ventilation through a tube inserted into a tracheostomy stoma, a patient undergoing general anesthesia, etc.

### BRIEF DESCRIPTION OF THE DRAWINGS

FIG. 1 is a longitudinal sectional view of a tracheal tube having volume inflators 10 and 20, which are inflated, according to a preferred embodiment of the present invention;

FIG. 2 is a longitudinal sectional view of a tracheal tube having a trachea sealing portion of a cross-shaped configuration according to a preferred embodiment of the present invention;

FIG. 3 illustrates a trachea sealing portion, which is supplied with a sealing medium 80, according to a preferred embodiment of the present invention;

FIG. 4 illustrates a cross-shaped trachea sealing portion, which is supplied with a sealing medium 80, according to a preferred embodiment of the present invention;

FIG. 5 is a sectional view of conduits, which supply the trachea sealing portion with a sealing medium 80, and sealing medium passage holes 30 according to a preferred embodiment of the present invention; and

FIG. 6 is an overall view of a tracheal tube having volume inflators 10 and 20, which are inflated, according to a preferred embodiment of the present invention.

### DESCRIPTION OF MAJOR REFERENCE NUMERALS IN THE DRAWINGS

10: first volume inflator
20: second volume inflator
30: sealing medium passage hole
31: volume inflation medium passage hole
40: first conduit
50: second conduit
60: volume regulator
70: volume provider
80: sealing medium
90: trachea wall
100: hollow tube

### DETAILED DESCRIPTION OF THE DISCLOSURE

Hereinafter, components and technical characteristics of the present invention will be described in more detail with reference to the following embodiments taken in connection with the accompanying drawings. However, it is to be noted that the following embodiments are only intended to illustrate aspects of the present invention, not limit the scope of the present invention.

### Embodiment 1

### Construction of Tracheal Tube

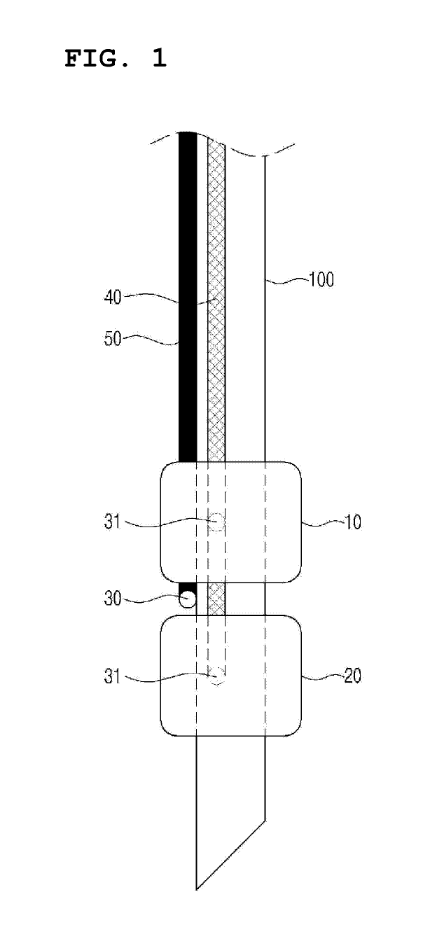

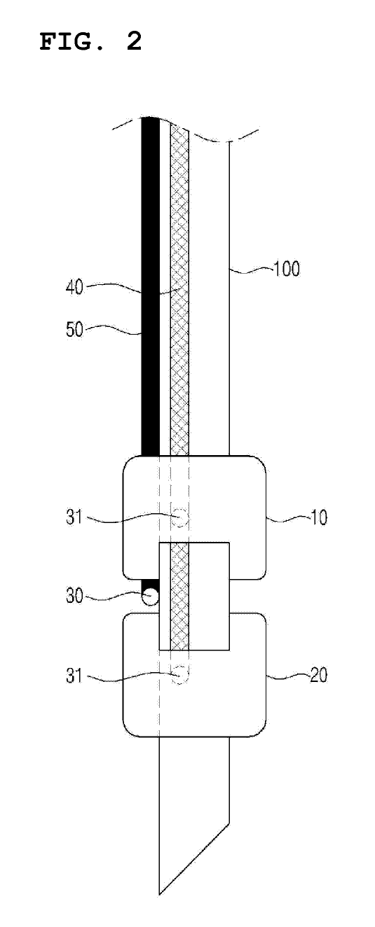

As shown in FIGS. 1 and 2, a tracheal tube according to the present invention includes a hollow tube 100 inserted into the trachea; a first volume inflator 10 surrounding one side of the hollow tube 100; a second volume inflator 20 positioned under the first volume inflator 10; a first conduit 40 adapted to inflate the first and second volume inflators 10 and 20; and a second conduit 50 adapted to supply a trachea sealing portion, which is positioned between the first and second volume inflators 10 and 20, with a sealing medium 80.

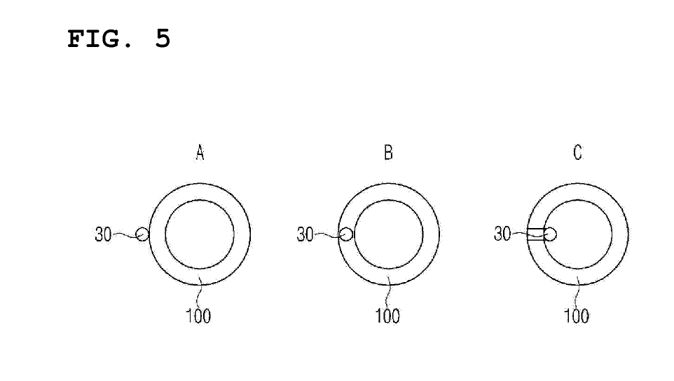

Although the second conduit 50 is shown in FIGS. 1 and 2 outside the hollow tube 100, the position is not limited thereto, and it may be positioned in a section of the hollow tube 100 or inside the hollow tube 100. If the second conduit 50 is positioned in a section of the hollow tube 100 or inside the hollow tube 100, a sealing medium passage hole 30 may be formed through the trachea sealing portion (FIG. 5).

In addition, as shown in FIG. 2, surfaces of the first and second volume inflators 10 and 20, which face each other, may be partially indented so that the trachea sealing portion has a cross-shaped configuration.

A tracheal tube according to a specific embodiment of the present invention includes a hollow tube 100 inserted into the trachea; a first volume inflator 10 surrounding one side of the hollow tube 100; a second volume inflator 20 positioned under the first volume inflator 10; a first conduit adapted to inflate the first volume inflator 10; a second conduit adapted to inflate the second volume inflator 20; and a third conduit adapted to supply a trachea sealing portion, which is positioned between the first and second volume inflators 10 and 20, with a sealing medium 80.

Although the third conduit is shown in FIGS. 1 and 2 outside the hollow tube 100, as described above, the position is not limited thereto, and it may be positioned in a section of the hollow tube 100 or inside the hollow tube 100. If the third conduit is positioned in a section of the hollow tube 100 or inside the hollow tube 100, a sealing medium passage hole 30 may be formed through the trachea sealing portion (FIG. 5).

The sealing medium 80 supplied to the trachea sealing portion may be, for example, VIASYS Electrolyte Gel (VIASYS healthcare), which is water-soluble gel having viscosity of 100-10.000 cPs, Sulridin lubrication jelly (Hanlim Pharm.), Lidocaine jelly (SungKwang Pharm.), K-Y jelly (Johnson & Johnson), K-Y ultra, Progel (Da Yo medical), or 50-1500 ml/min of air or oxygen fluid.

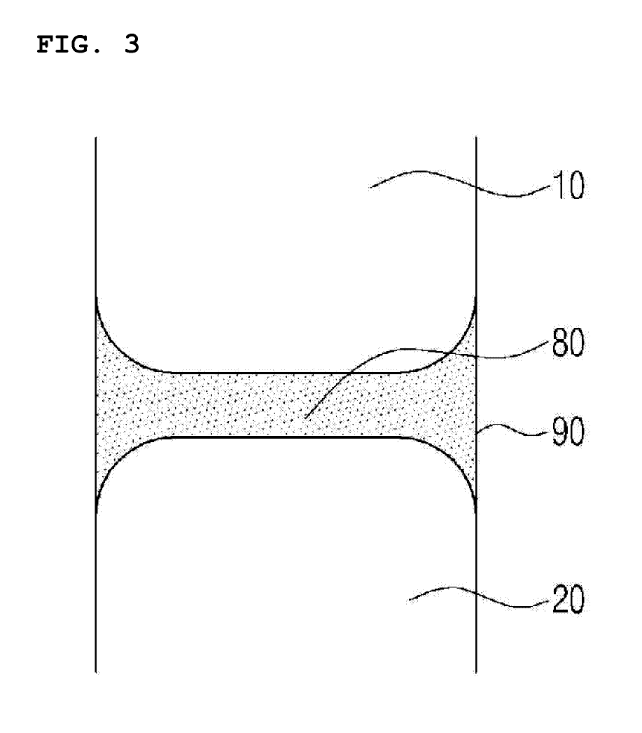

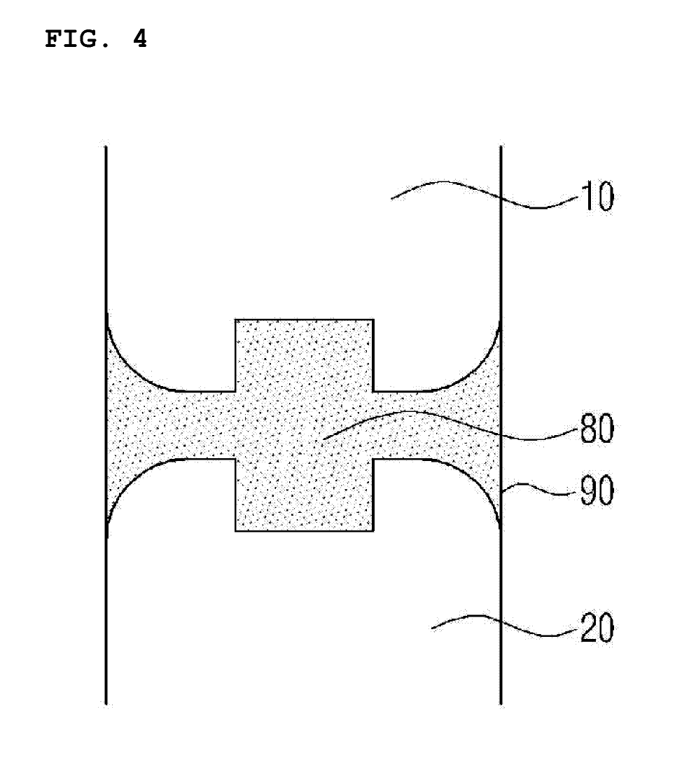

FIGS. 3 and 4 illustrate trachea sealing portions of a trachea tube inserted into the trachea, which are supplied with a sealing medium 80.

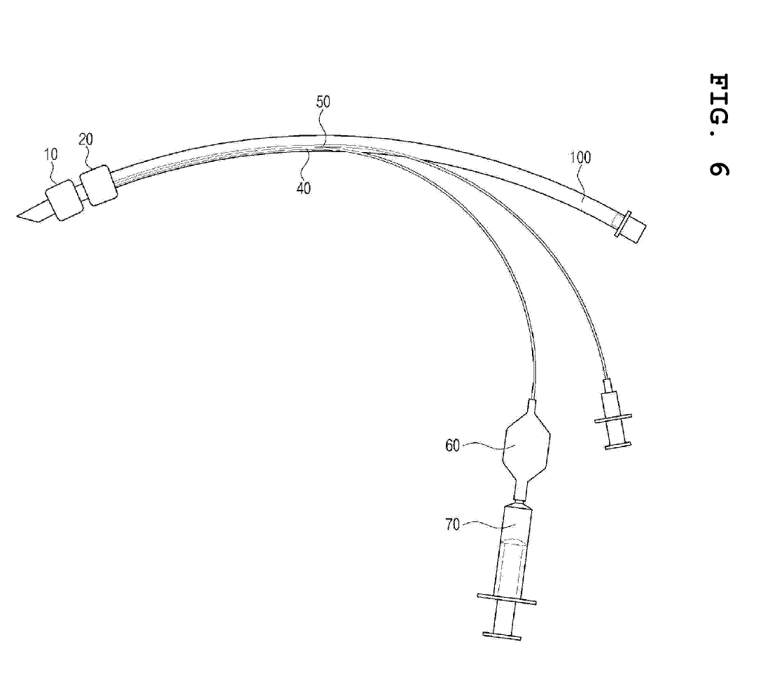

The tube may further include a volume controller, on the other side, adapted to inflate the volume of the first and second volume inflators 10 and 20 (FIG. 6).

The tube may have a volume inflation medium passage hole 31 formed through one side so that the volume inflation medium can flow from the volume controller to the first and second volume inflators 10 and 20.

In a specific embodiment, the volume inflators are cuffs.

In a specific embodiment, the volume inflators may have a cylindrical shape. During volume inflation, the volume inflators may have a diameter within the range of 0.5-3.0 cm. In addition, during volume inflation, the volume inflators may have a length within the range of 0.5-3.0 cm.

In a specific embodiment, the volume inflation medium may be gas or liquid. For example, the gas may be air, and the liquid may be water (or saline solution).

In a specific embodiment, the volume controller may include a volume regulator 60 adapted to regulate the direction of flow of the volume inflation medium, which flows through the first conduit, and a volume provider 70 adapted to introduce the volume inflation medium through the first conduit.

In a specific embodiment, the volume provider 70 may be a syringe.

In a specific embodiment, the tracheal tube according to the present invention may be disposable.

### Embodiment 2

### Function of Tracheal Tube

A tracheal tube according to the present invention is inserted into a model trachea that simulates a patient's larynx. The volume of dual volume inflators (cuffs) is inflated by using the volume controller, and the trachea sealing portion is supplied with a sealing medium 80. When the dual volume inflators are inflated to have a pressure of 20 cmH₂O, and the trachea sealing portion, which is formed between them, is provided with gel (VIASYS, Madison, Wis., USA) until the pressure of the double volume inflators reaches 25 cmH₂O (average volume of 2.4 ml), all of conventional tubes (Standard endotracheal tube° Euromedical, Malaysia; Safety-Flex™, Mallinckrodt, Athlone, Ireland) result in leakage within one minute, but none of the five tracheal tubes according to the present invention result in leakage even after two weeks. Pressure applied to the model trachea during this period is in the range of 7-20 cmH₂O, which is not high enough to damage the trachea. When the tubes are removed, they carry almost all gel, and the amount of gel remaining on the model trachea is 0.016 ml on average, which corresponds to about 0.6% of the initial amount.

Furthermore, when the volume inflators are inflated and 50-1500 ml/min of oxygen is supplied, tracheal tubes according to the present invention are also free from leakage.

While the invention has been described in connection with various aspects, it will be understood that the invention is capable of further modifications. This application is intended to cover any variations, uses or adaptation of the invention following, in general, the principles of the invention, and including such departures from the present disclosure as come within the known and customary practice within the art to which the invention pertains.

1. A tracheal tube comprising:
a hollow tube inserted into trachea;
a first volume inflator surrounding one side of the hollow tube;
a second volume inflator positioned under the first volume inflator;
a first conduit adapted to inflate the first and second volume inflators; and
a second conduit adapted to supply a sealing medium to a trachea sealing portion positioned between the first and second volume inflators.

2. A tracheal tube comprising:
a hollow tube inserted into trachea;
a first volume inflator surrounding one side of the hollow tube;
a second volume inflator positioned under the first volume inflator;
a first conduit adapted to inflate the first volume inflator;
a second conduit adapted to inflate the second volume inflator; and
a third conduit adapted to supply a sealing medium to a trachea sealing portion positioned between the first and second volume inflators.

3. The tracheal tube as claimed in claim 1, further comprising a volume controller positioned on the other side of the tube and adapted to inflate the volume of the first or second volume inflator.

4. The tracheal tube as claimed in claim 2, further comprising a volume controller positioned on the other side of the tube and adapted to inflate the volume of the first or second volume inflator.

5. The tracheal tube as claimed in claim 3, wherein a volume inflation medium passage hole is formed through one side of the tube so that a volume inflation medium supplied from the volume controller can flow into the first or second volume inflator.

6. The tracheal tube as claimed in claim 4, wherein a volume inflation medium passage hole is formed through one side of the tube so that a volume inflation medium supplied from the volume controller can flow into the first or second volume inflator.

7. The tracheal tube as claimed in claim 1, wherein the first or second volume inflator is a cuff.

8. The tracheal tube as claimed in claim 2, wherein the first or second volume inflator is a cuff.

9. The tracheal tube as claimed in claim 1, wherein the first or second volume inflator has a cylindrical shape.

10. The tracheal tube as claimed in claim 2, wherein the first or second volume inflator has a cylindrical shape.

11. The tracheal tube as claimed in claim 7, wherein, during volume inflation, the first or second volume inflator has a diameter of 0.5-3.0 cm.

12. The tracheal tube as claimed in claim 8, wherein, during volume inflation, the first or second volume inflator has a diameter of 0.5-3.0 cm.

13. The tracheal tube as claimed in claim 7, wherein, during volume inflation, the first or second volume inflator has a length of 0.5-3.0 cm.

14. The tracheal tube as claimed in claim 8, wherein, during volume inflation, the first or second volume inflator has a length of 0.5-3.0 cm.

15. The tracheal tube as claimed in claim 1, wherein the interval between the first and second volume inflators is 0.2-1.0 cm.

16. The tracheal tube as claimed in claim 2, wherein the interval between the first and second volume inflators is 0.2-1.0 cm.

17. The tracheal tube as claimed in claim 3, wherein the volume controller comprises:
a volume regulator adapted to regulate the flow direction of the volume inflation medium introduced through the first conduit; and
a volume provider adapted to introduce the volume inflation medium along the first conduit.

18. The tracheal tube as claimed in claim 4, wherein the volume controller comprises:
a volume regulator adapted to regulate the flow direction of the volume inflation medium introduced through the first conduit; and
a volume provider adapted to introduce the volume inflation medium along the first conduit.

19. The tracheal tube as claimed in claim 17, wherein the volume provider is a syringe.

20. The tracheal tube as claimed in claim 18, wherein the volume provider is a syringe.

21. The tracheal tube as claimed in claim 1, wherein a volume inflation medium provided through the first conduit is gas or liquid.

22. The tracheal tube as claimed in claim 2, wherein a volume inflation medium provided through the first or second conduit is gas or liquid.

23. The tracheal tube as claimed in claim 1, wherein the sealing medium provided through the second conduit is gel, jelly, or gas.

24. The tracheal tube as claimed in claim 2, wherein the sealing medium provided through the third conduit is gel, jelly, or gas.

25. The tracheal tube as claimed in claim 1, wherein the second conduit is positioned outside the hollow tube, in a section of the hollow tube, or inside the hollow tube.

26. The tracheal tube as claimed in claim 2, wherein the third conduit is positioned outside the hollow tube, in a section of the hollow tube, or inside the hollow tube.

27. The tracheal tube as claimed in claim 25, wherein, when the second conduit is positioned in a section of the hollow tube or inside the hollow tube, a sealing medium passage hole is formed through the trachea sealing portion.

28. The tracheal tube as claimed in claim 26, wherein, when the third conduit is positioned in a section of the hollow tube or inside the hollow tube, a sealing medium passage hole is formed through the trachea sealing portion.

29. A method for manufacturing a tracheal tube, the method comprising the steps of:
(i) preparing a hollow tube;
(ii) positioning a first volume inflator on one side of the hollow tube;
(iii) positioning a second volume inflator under the first volume inflator;
(iv) positioning a first conduit inside the hollow tube, the first conduit being adapted to inflate the first and second volume inflators; and
(v) positioning a second conduit adapted to supply a sealing medium to a trachea sealing portion between the first and second volume inflators.

30. A method for manufacturing a tracheal tube, the method comprising the steps of:
(i) preparing a hollow tube;
(ii) positioning a first volume inflator on one side of the hollow tube;
(iii) positioning a second volume inflator under the first volume inflator;
(iv) positioning a first conduit inside the hollow tube, the first conduit being adapted to inflate the first volume inflator;
(v) positioning a second conduit inside the hollow tube, the second conduit being adapted to inflate the second volume inflator; and
(vi) positioning a third conduit adapted to supply a sealing medium to a trachea sealing portion between the first and second volume inflators.

31. The method as claimed in claim 29, further comprising a step of positioning a volume controller on the other side of the tube, the volume controller being adapted to inflate the volume of the first or second volume inflator.

32. The method as claimed in claim 30, further comprising a step of positioning a volume controller on the other side of the tube, the volume controller being adapted to inflate the volume of the first or second volume inflator.

33. The method as claimed in claim 29, wherein a volume inflation medium passage hole is formed through one side of the tube so that a volume inflation medium supplied from the volume controller can flow into the first or second volume inflator.

34. The method as claimed in claim 30, wherein a volume inflation medium passage hole is formed through one side of the tube so that a volume inflation medium supplied from the volume controller can flow into the first or second volume inflator.

35. The method as claimed in claim 29, wherein, in step (v), the second conduit is positioned outside the hollow tube, in a section of the hollow tube, or inside the hollow tube.

36. The method as claimed in claim 30, wherein, in step (vi), the third conduit is positioned outside the hollow tube, in a section of the hollow tube, or inside the hollow tube.

37. The method as claimed in claim 35, wherein, when the second conduit is positioned in a section of the hollow tube or inside the hollow tube, a sealing medium passage hole is formed through the trachea sealing portion.

38. The method as claimed in claim 36, wherein, when the third conduit is positioned in a section of the hollow tube or inside the hollow tube, a sealing medium passage hole is formed through the trachea sealing portion.

39. A method for preventing endotracheal aspiration by providing a medium through the first and second conduits of the tracheal tube as claimed in claim 1.

40. The method as claimed in claim 39, wherein a volume inflation medium provided through the first conduit is gas or liquid.

41. The method as claimed in claim 39, wherein a sealing medium provided through the second conduit is gel, jelly, or gas.

42. A method for preventing endotracheal aspiration by providing a medium through the first, second, and third conduits of the tracheal tube as claimed in claim 2.

43. The method as claimed in claim 42, wherein the medium provided through the first or second conduit is gas or liquid.

44. The method as claimed in claim 42, wherein the medium provided through the third conduit is gel, jelly, or gas.
